(19) United States
(12) Patent Application Publication
(10) Pub. No.: US 2006/0058392 A1
(43) Pub. Date: Mar. 16, 2006

(54) Use of a rhein in a therapeutic treatment requiring a rise in the rate of heme oxygenase

(76) Inventors: Suzy Charbit, Creteil (FR); Francois Schutze, St-Nom-la-Breteche (FR); Diego Provvedini, Bougival (FR); Herve Ficheux, Nogent-sur-Marne (FR)

Correspondence Address: E Joseph Gess;Burns Doane Swecker & Mathis, P O Box 1404, Alexandria, VA, 22313-1404, US

(21) Appl. No.: 10/522,035

(22) PCT Filed: Jul. 18, 2003
(86) PCT No.: PCT/FR03/02286

§ 371 Date: Sep. 29, 2005

(30) Foreign Application Priority Data
Jul. 23, 2002 (FR) 02/9340

(51) Int. Cl. A61K 31/192; A61K 31/225

(52) U.S. Cl. 514/569; 514/548

(57) ABSTRACT

The invention concerns a novel therapeutic application of rhein and diacerhein. Rhein and diacerhein, as well as the salts and esters thereof, can be used for treating diseases requiring a rise in the rate of the heme oxygenase enzyme, providing prevention and inhibition of stress on cells and tissues, and treatment and prevention of organ and tissue transplant rejections.

The present invention concerns the treatment in human or animal therapeutics of affections requiring an increase in heme oxygenase enzyme levels, through the administration of an efficient dose of rhein or diacerein or of one of their salts or esters, as well as the use of rhein or diacerein or of one of their salts or esters for the manufacture of a medicinal product for the treatment of diseases requiring an increase in heme oxygenase enzyme levels, by acting on the causes of some acute and chronic conditions, by ensuring the prevention and the inhibition of the effects of stress on cells and tissues, and by ensuring the prevention and the treatment of organ and tissue transplant rejection.

The reaction of the immune system is a physiopathologic mechanism of response to several types and forms of aggression which aim at the organism (<<stress >>). This reaction may be responsible for several important pathological forms, referenced for example in Harrison's Principles of Internal Medicine, 14^{th} edition (1998), 749-754. A great number of studies have been directed to the mediators responsible for the onset of the reaction of the immune system elements, and to the pharmacological and therapeutic control of this reaction during its initial phases. These studies have allowed for the production and the marketing of efficient medicinal products, for example in the treatment of acute inflammation.

Under normal conditions, the protection and response mechanisms of the organism to stress (chemical, thermal, mechanical, infectious, etc.) can take control of the causes that are at the basis of the reaction and stop the process. Indeed, researchers have often found that the reaction of the immune system elements could subside during a second phase after an acute initial phase. However, few studies have addressed the issue of the spontaneous benign progression of this reaction and of its pharmacological and therapeutic control.

Because of the persistence of the causes, and/or of the inefficacy of the treatment, the reaction of the immune system elements may become chronic. The progression to the chronic state is frequent and may take different important pathological aspects, referenced notably in Tarkowski A. and al. Mol. Med. Today (1988) 4:15-18; and Levy B. D. and al., Nature Immunol. (2001) 2:612-619. The problematic related to this progression to the chronic state is at the heart of many research projects. However, satisfactory treatments available nowadays are few. Besides, these treatments are often ill tolerated by the patients and may cause serious adverse events, even at low doses.

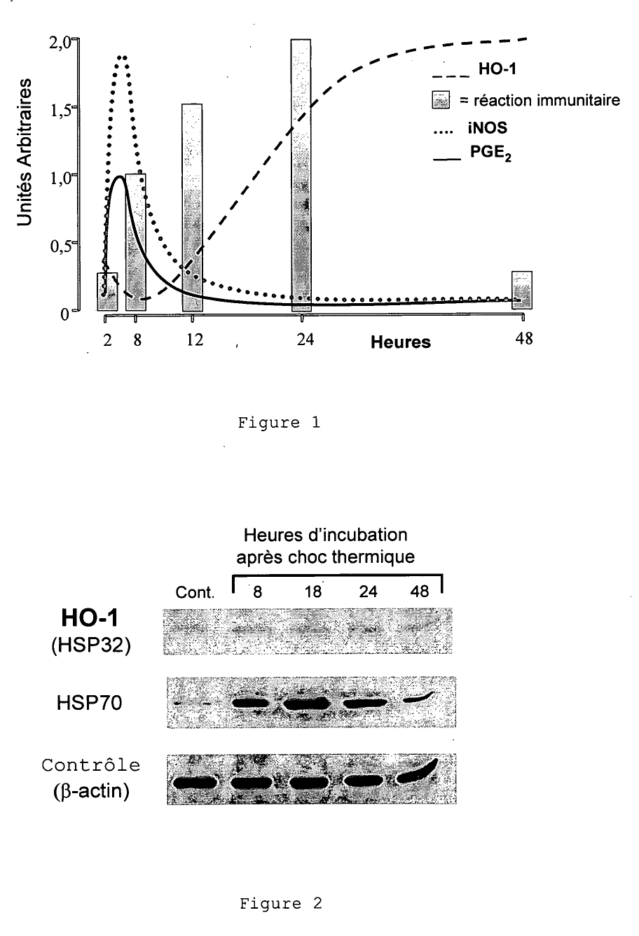

Some works have shown the influence of certain endogen factors on the spontaneous benign progression of the reaction of the immune system elements. D. Willis and al. (Nature Medicine (1996) 2: pp. 87-90) have thus shown that an enzyme, heme oxygenase-1 (HO-1), is progressively expressed in the most advanced phases of the acute inflammatory reaction, and precedes the resolution phase of this reaction. A coherent model of the main events occurring during this reaction is thus illustrated on FIG. 1 (model of induced pleurisy after injection of carrageen in the rat), in which “HO-1” represents the levels of heme oxygenase-1, “iNOS” the inducible nitric-oxide synthetase, and “PGE₂” prostaglandin E₂. It can be observed that the levels of heme oxygenase increase progressively with time and precede the decrease of the reaction of the immune system elements.

The heme oxygenase enzyme belongs to the class of “heat-shock proteins” (HSP): it is also known as “heat-shock protein 32K” (HSP32)(see Keyse S. M. and al., Proc. Natl. Acad. Sci. USA (1991) 86:99-103). HSPs belong to the family of proteins whose expression is stimulated by stress (heat, hypoxia, oxidation, intoxication by metals, etc.), as verified by experiments carried out by the applicant in vitro on mice macrophage cultures, human chondrocytes and isolated cartilage of rat femoral head. FIG. 2 shows the results of a “Western blot” (a sensitive method of measurement of the levels of protein of interest) which analyses the expression of HSP32 (HO-1) and HSP70 after thermal shock (1 hour at 43° C.) in the model of rat femoral head cartilage (Cont.=Control).

HSPs play a very important part in the defence and cellular repair mechanisms related to stress. In particular, HO-1 has a very important modulating effect during the inflammatory response: the inflammation is suppressed as the enzyme levels increase, whereas an inhibition of the enzyme causes an increase of the inflammatory response (Nature Medicine (1996) 2:87-90).

The discovery of HO-1 and the appreciation of its effects suggested the possibility of a pharmacological and therapeutic control of the cellular response to stress; indeed, it would be advantageous to dispose of medicinal products likely to favour and/or maintain the increase of the level of enzyme HO-1, for use in the treatment of several conditions which imply cellular and tissular stress, such as has been observed in immunosuppressed patients, notably.

Besides, the cellular mechanisms responsible for the response to stress also act during the phenomena observed as a response to tissue or organ transplant (Harrison's Principles of Internal Medicine, 14^{th} edition (1998), 1760-68). Therefore, it would also be advantageous to dispose of medicinal products likely to favour the increase of the levels of enzyme HO-1 to protect the organism against the degradation of the graft observed during tissue and organ transplants, and useful in the prevention and the treatment of transplant rejections.

Rhein and some of its derivatives, including diacerein, have been used in human and veterinary therapeutics as active substances of medicinal products. Methods of preparation have been developed to obtain diacerein at a good yield and of purity compatible with its use in therapeutics, presenting a very low content in aloemodin and other undesired impurities.

In human therapeutics, diacerein has been administered to patients presenting with osteoarthritis and experiencing pain and difficulties to move. Besides, the treatment by diacerein slows down the progression of osteoarthritis, with a good safety of use. However, diacerein and rhein both have a moderate antalgic and anti-inflammatory symptomatic activity in the acute phase of osteoarthritis (Nguyen and al., Arthritis and Rheumatism (1994) 37:529-536).

Now, the works and experiments performed by the applicant and aiming at clarifying the action mechanism of diacerein and rhein have unexpectedly shown that rhein, diacerein and their salts or esters significantly promote the formation of HO-1, and favour the control mechanism of the cellular and tissular response to stress. It was therefore possible to demonstrate that the administration of rhein, diacerein or their salts or esters protects the organism against stress-related deleterious events.

Besides, the administration of rhein, diacerein, or their salts or esters also protects the organism against organ or tissue degradation, and notably cartilage degradation, by the immune system cells, and controls the phenomena arising as a response to organ and tissue transplants, which makes it possible to envisage their use in the prevention and treatment of organ and tissue graft rejections.

The present invention therefore relates to the use of diacerein, and more generally of rhein and of the rhein derivatives, in human and veterinary therapeutics in the treatment of affections requiring high levels of the heme oxygenase enzyme, in the prevention and inhibition of the effects of stress on cells and tissues, and for the prevention and treatment of organ and tissue graft rejections.

The invention also concerns the use of rhein and the rhein derivatives, in particular diacerein, for the manufacture of a medicinal product for the treatment of a condition requiring a high level of heme oxygenase.

Rhein and diacerein, together with their salts or esters, therefore appear to be particularly advantageous to favour or maintain a high level of heme oxygenase, in the prevention and the inhibition of the effects of stress on cells and tissues, and in the prevention and treatment of organ and tissue graft rejections in subjects requiring such treatment.

Rhein and the rhein derivatives that can be used in this invention, notably diacerein, can be represented by the following general formula (I): 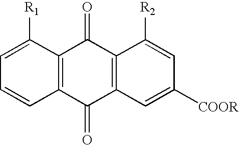
in which R represents a hydrogen atom, or an alkyl group, for example a methyl, ethyl or propyl group, or an alkaline or earth-alkaline metal atom, for example an atom of sodium, potassium or calcium, R₁ and R₂, identical or different, represent a hydroxy group or an acyloxy group of formula R′ —COO— in which R′ is an alkyl group of 1 to 4 carbon atoms, for example a methyl, ethyl or isopropyl group.

In the above general formula (I), R preferably represents a hydrogen atom, and R₁ and R₂ preferably represent a hydroxy or acetoxy group. The above general formula (I) in which R is a hydrogen atom and R₁ and R₂ are an acetoxy group —COOCH₃ is that of diacerein.

Diacerein and rhein can be prepared according to the known methods of the technique, and for example from aloe or sena leaf extraction products, such as sennosides, or by barbaloin acetylation followed by chromium oxide oxidation. The synthesis processes described in patents EP 801639 and EP 909268 can also be used. For example, these processes consist in a Diels-Alder reaction on a naphtoquinone such as juglone using an acyclic diene to obtain tetrahydroanthraquinone which can easily be transformed into rhein and diacerein after oxidising deprotection.

The diacerein obtained thanks to these processes can be purified if necessary to obtain a product that perfectly complies with pharmaceutical standards and offers all the guarantees desired. For example, the purification process described in patent EP 754173 can be used, according to which a soluble diacerein salt is prepared by action of triethylamine and potassium acetate, followed by hydrolysis in slightly acid medium.

The studies carried out by the applicant to evidence the effect of diacerein, rhein and their salts or esters on HO-1 have been conducted using validated models, as indicated here-after.

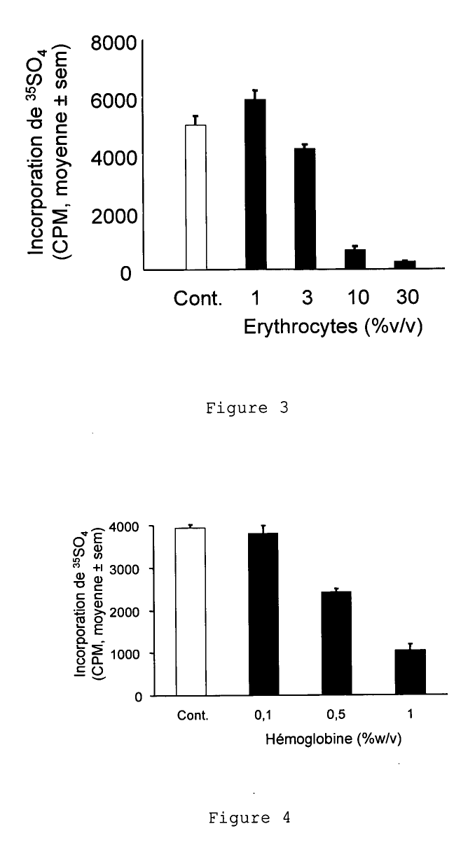

The in vitro works and experiments have used the following models:
1. A validated model of tissue culture, and notably rat femoral head cartilage, treated with erythrocytes (in % w/v), erythrocyte lysate (3×10⁶), or haemoglobin (in % w/v), without and after thermal stress (1 h at 43° C.). The whole cartilage was measured by incorporating radioactive sulphate (³⁵SO₄; in coups per minutes, CPM). In this model, the erythrocytes, the erythrocyte lysate and haemoglobin cause cellular destruction, as demonstrated by a diminution of the cellular viability and of the incorporation of the radioactive sulphate by the cells, as shown on FIGS. 3 and 4.

FIG. 3 shows that the cellular destruction caused by the erythrocytes is dose dependent, as evidenced by the diminution of the cellular incorporation of radioactive sulphate (model: rat femoral head cartilage with erythrocytes in % v/v; Cont.=control; erythrocyte-free solution; ³⁵SO₄=radioactive sulphate; sem=standard error).

Similarly, FIG. 4 shows that the cellular destruction caused by haemoglobin is dose dependent, as evidenced by the diminution of the cellular incorporation of radioactive sulphate (model: rat femoral head cartilage with haemoglobin in % v/v; Cont.=control; haemoglobin-free solution; ³⁵SO₄=radioactive sulphate; sem=standard error).

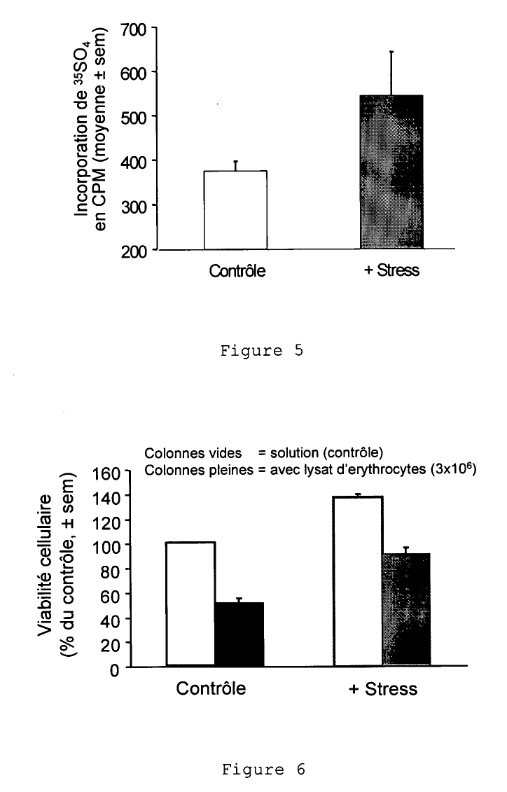

In this model of tissue culture, HSPs which have previously been induced by stress prevent cellular destruction, as demonstrated on FIG. 5 by an increase of the incorporation of radioactive sulphate by the cells (model: rat femoral head cartilage under thermal stress (1 h at 43° C.) and then treated with haemoglobin (1% w/v); ³⁵SO₄=radioactive sulphate; sem=standard error).
2. A validated model of cell culture, and notably human chondrocytes, treated with erythrocytes (in % w/v), erythrocyte lysate (3×10⁶), or haemoglobin (in % w/v), without and after thermal stress (1 h at 43° C.). The whole cellular culture was measured by its cellular viability (live cells compared to the control), the presence of apoptosis (nuclear fragmentation; in number and percentage compared to the control), and the incorporation of radioactive sulphate (³⁵SO₄; in coups per minutes, CPM).

As was already observed with the femoral head cartilage, the erythrocytes, erythrocyte lysate and haemoglobin also cause cellular destruction in this model. However, stress-induced HSPs prevent this cellular destruction and preserve the cells, as shown on FIG. 6 which counts the live cells (model: human chondrocytes under thermal stress (1 h at 43° C.) and treated with erythrocyte lysate (3×10⁶); sem=standard error).

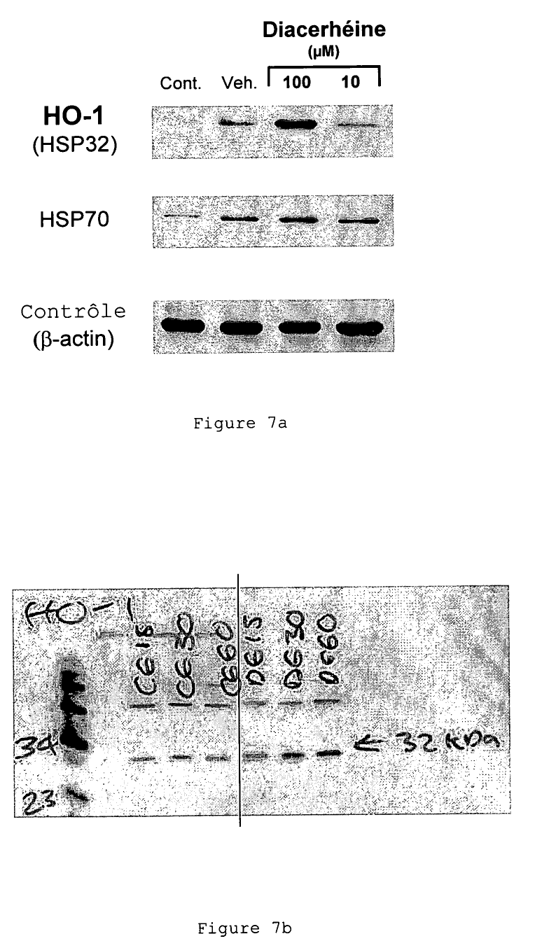

Based on these experiments, the studies carried out by the applicant evidenced the effects of diacerein, rhein and their salts or esters on the expression of HO-1, as illustrated in detail here-after as a reference to FIGS. 7a, 7b and 7c.

FIG. 7a is a <<Western blot >> (model: rat femoral head cartilage; Cont.=control; Veh.=reference solution; diacerein: 10 μM and 100 μM) which shows that diacerein, when compared to the control and the reference solution, triggers a dose-dependent increase of the expression of HO-1.

FIGS. 7b and 7c are also <<Western blots >> of cells (model: mice macrophages) cultivated with and without diacerein (FIG. 7b: columns “Dε” and “Cε” respectively) or rhein (FIG. 7c) at a concentration of 10⁻⁵ M, and later analysed at 15, 30 and 60 minutes (FIG. 7b) and at 0, 15, 30, 60, 120 minutes as well as 18 hours (FIG. 7c). The identity of the protein of interest, HO-1, was confirmed as regards its molecular mass (first column on the left in the Figures). These results demonstrate that diacerein and rhein trigger a time-dependant increase of the expression of HO-1.

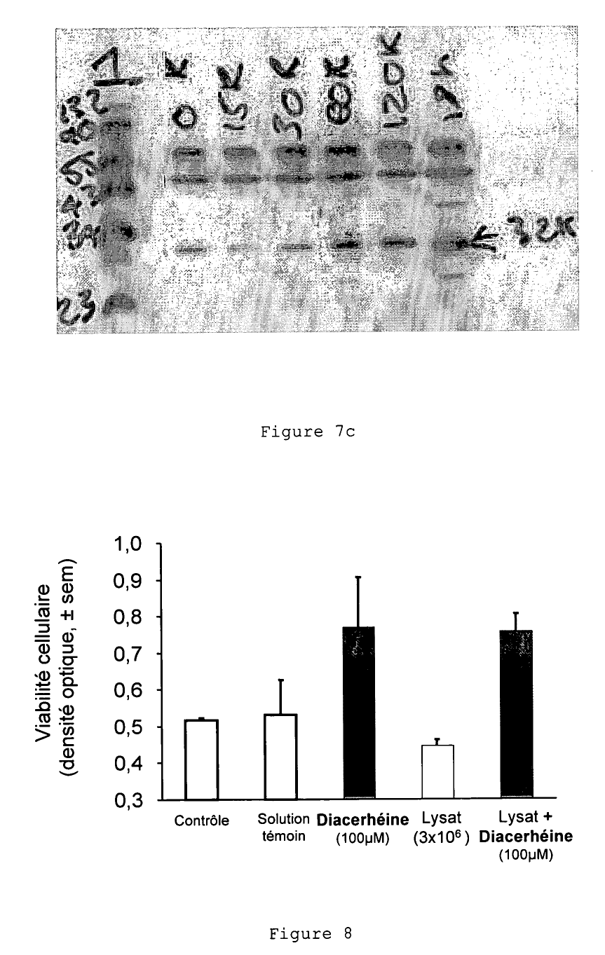

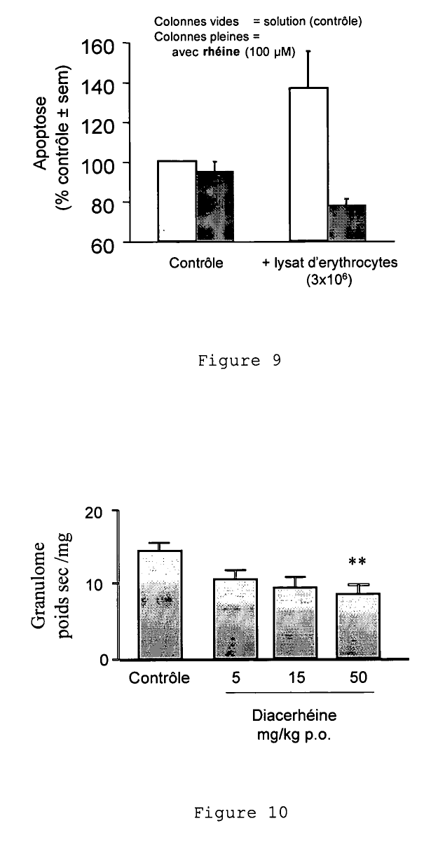

Using the in vitro models described here-above, the studies conducted by the applicant also demonstrated that diacerein, rhein and their salts or esters, preserve cellular integrity thanks to their effect on HO-1, as illustrated in detail in FIGS. 8 and 9.

FIG. 8 (model: human chondrocytes; lysate=product of the lysis of 3×10⁶ of erythrocytes; diacerein: 100 μM; sem=standard error) shows that treatment with diacerein prevents the cellular destruction caused by the erythrocyte lysate.

FIG. 9 (model: human chondrocytes; lysate=product of the lysis of 3×10⁶ of erythrocytes; rhein: 100 μM; sem=standard error) shows that treatment with rhein prevents the apoptosis caused by the erythrocyte lysate.

The main conclusions of the works and experiments conducted by the applicant in vitro with the models described here-above, on the effects of diacerein, rhein and their salts or esters, are the following:
Diacerein, rhein and their salts or esters induce a dose-dependent increase of the levels of the HO-1 enzyme which plays a part in the protection of cellular integrity;
Thanks to this increase in HO-1, diacerein, rhein and their salts or esters preserve the cell and tissue integrity when confronted to stress-induced deleterious reactions;
Diacerein, rhein and their salts or esters prevent apoptosis (fragmentation of the nucleus, followed by the destruction and death of the cells).

The applicant also conducted works and experiments in vivo, using the model described here-after.

The effect of diacerein, rhein and their salts or esters on the intensity of the tissular reaction and the destruction and rejection phenomena was assessed following a tissue implantation, using the model of induced granuloma and tissue degradation of rat tissue in mice (Bottomley K M and al., Osteoarthritis and Cartilage (1998) 6: 19-23.).

The method comprises the following steps:
The isolated tissue (Wistar rat, male) are placed in sterile cotton and implanted subcutaneously in TO mice;
the subcutaneous implants are kept for a period of 2 weeks;
daily treatment of the animals with diacerein, rhein, their salts or esters, as well as with the controls and comparatives, at the optimum doses indicated;
collection of the implanted tissue and of the reactive granuloma;
measurement of the degree of tissular inflammatory response (assessment of the dimensions of the granuloma, of the number of inflammatory cells in the exudate and of the volume of the exudate), and of the rejection (assessment of the degradation of the implanted tissue).

Using the model described here-above, the studies conducted by the applicant demonstrated that diacerein, rhein and their salts or esters reduce the tissular reaction (formation of the granuloma, recruitment of inflammatory cells, formation of an exudate) caused by the transplant of rat tissue to the mouse, and preserve the integrity of the implanted tissue, as illustrated in detail on FIGS. 10, 11 and 12 here-after.

FIG. 10 shows that the treatment with diacerein causes a dose-dependent reduction of the tissular reaction (formation of the reactive granuloma) (diacerein: 5, 15 and 50 mg/kg, per oral route; ** statistically significant difference compared to the control: p<0.01).

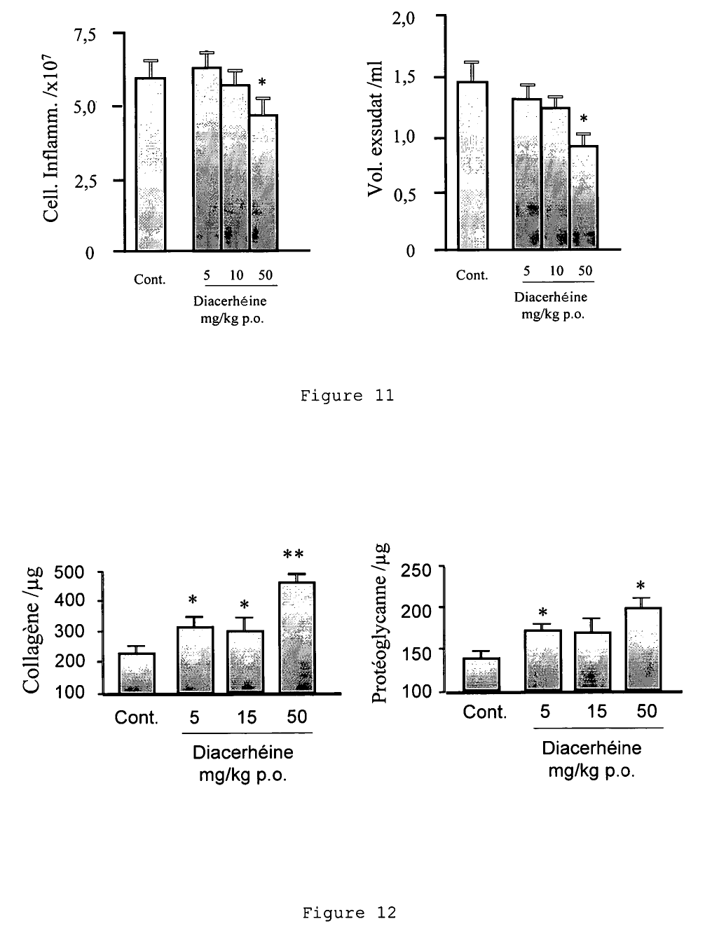

FIG. 11 shows that the tissular reaction triggered by the implantation of rat tissue to the mouse is reduced in a dose-dependent manner by treatment with diacerein (on the left on FIG. 11: number of inflammatory cells; on the right: volume of the exudate) (Cont.=control; diacerein: 5, 15 and 50 mg/kg, per oral route; * statistically significant difference compared to the control: p<0.05).

FIG. 12 shows that treatment with diacerein preserves the integrity of the grafted tissue according to the dose of diacerein administered, as evidenced by the conservation of the content in collagen (on the left on FIG. 12) and in glycosamino-glycan (GAG; on the right) of the grafted tissue (diacerein: 5, 15 and 50 mg/kg, per oral route; statistically significant difference versus the control: * p<0.05: ** p<0.01).

The main conclusions to the works and experiments conducted by the applicant on the effects of diacerein, rhein, and their salts or esters in vivo with the model described here-above are the following:
diacerein, rhein, and their salts or esters reduce the tissular reaction (dimension of the granuloma, number of inflammatory cells, volume of the exudate) caused by the implantation of rat tissue to the mouse;
diacerein, rhein, and their salts or esters preserve the integrity of the grafted tissues when confronted to the tissular reaction;
diacerein, rhein, and their salts or esters prevent the destruction and rejection of the grafted tissue.

For the treatment of acute inflammatory phenomena, the medicinal products that are most widely used are non steroidal anti-inflammatory drugs (NSAID), even if they present certain adverse events, and in particular a tendency to induce gastric and intestinal ulcers (Goodman and Gilman, The Pharmacological Basis of Therapeutics; 9^{th} edition, McGraw Hill). These adverse events are related to the inhibition of the cyclo-oxygenase-1 enzyme (COX-1; constitutive isoform).

The discovery of the existence of another isoform of the cyclo-oxygenase enzyme, cyclo-oxygenase-2 (COX-2; isoform induced in the inflammation onset), opened new perspectives towards the development of potentially more specific and safer dugs. Currently available NSAIDs act as selective inhibitors of the action of COX-2, and therefore act on the inflammation, causing less adverse events related to the upper gastro-intestinal tractus. A new class of drugs, the COX-2 inhibitors such as celecoxib and rofecoxib, was thus developed for the symptomatic treatment of inflammatory diseases.

However, no favourable effect was observed so far on the heme oxygenase enzyme whether for the <<traditional >> NSAIDs or for the COX-2 inhibitors.

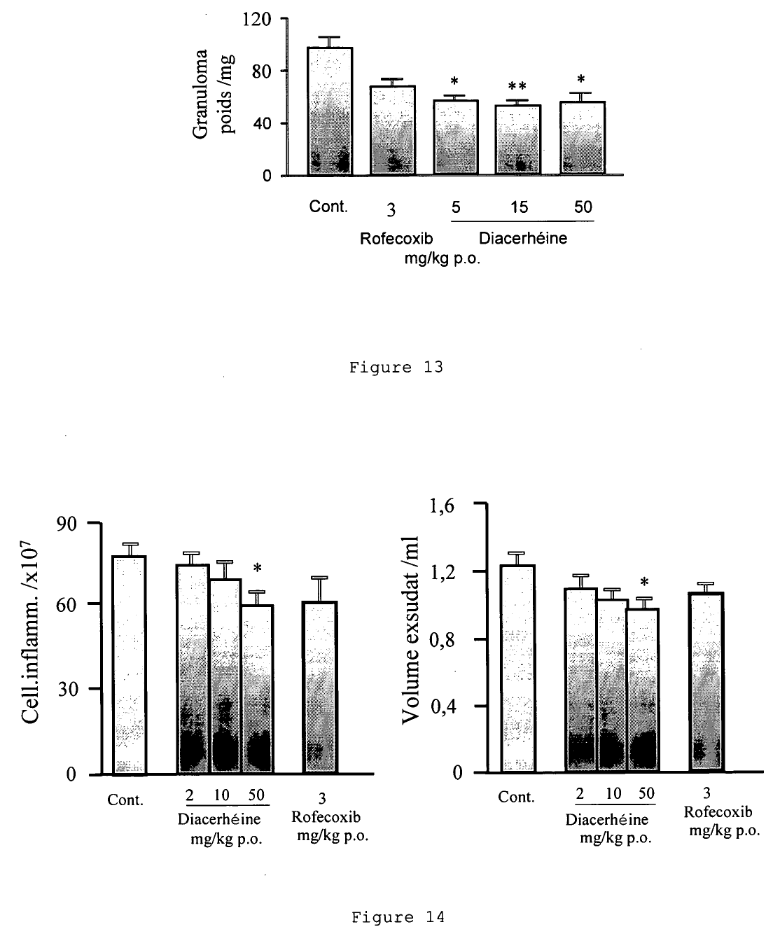

The studies conducted by the applicant confirmed that traditional NSAIDs as well as COX-2 inhibitors reduce the tissular reaction (granuloma, inflammatory cell infiltration, exudate) caused by the transplant of rat tissue to the mouse. However, neither the traditional NSAIDs nor the COX-2 inhibitors preserve the integrity of the grafted tissue, contrarily to rhein and diacerein, as shown on the attached FIGS. 13, 14 and 15.

FIG. 13 shows the comparison of the effects of treatment with a COX-2 inhibitor (rofecoxib) on the one hand, and diacerein on the other hand, on the reduction of the tissular reaction (formation of a reactive granuloma) caused by the implantation of rat tissue in the mouse (diacerein: 5, 15 and 50 mg/kg, rofecoxib: 3 mg/kg, per oral route; statistically significant difference compared to the control: p<0.05; ** p<0.01).

FIG. 14 allows for the comparison of the effects of a treatment with rofecoxib on the one hand, and diacerein on the other hand, on the reduction of the tissular reaction caused by the implantation of rat tissue in the mouse. The graph on the left illustrates the cellular infiltration, and the one on the right the volume of the exudates (Cont.=control; DAR: diacerein: 2, 10 and 50 mg/kg, rofecoxib: 3 mg/kg, per oral route; statistically significant difference compared to the control: * p<0.05).

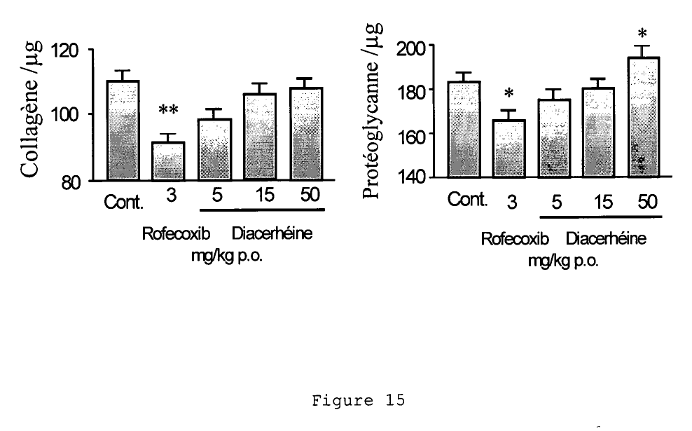

FIG. 15 allows for the comparison of the differences between the effect of treatment with rofecoxib on the one hand, and diacerein on the other hand, on the preservation of the integrity of the grafted tissue. The graph on the left corresponds to the content in collagen, the one on the right to the content in glycosaminoglycan (GAG) of the tissue (diacerein: 5, 15 and 50 mg/kg, rofecoxib: 3 mg/kg, per oral route; statistically significant difference compared to the control: *p<0.05: **p<0.01).

The results obtained show that the properties of diacerein and rhein are significantly different from those of other drugs, such as traditional non-steroidal anti-inflammatory drugs (NSAIDs) and COX-2 inhibitors, notably regarding the preservation of the grafted tissue and the rejection of this tissue.

According to the present invention, it may be useful to associate diacerein, or rhein, or their salts or esters, with a traditional NSAID or a COX-2 inhibitor, the actions of which may be complementary, according to the pathology being treated. Diacerein may thus be associated with an NSAID such as diclofenac at a dose comprised between 25 and 150 mg per day, or with a COX-2 inhibitor such as rofecoxib at a dose comprised between 10 and 50 mg per day.

As previously indicated, diacerein, rhein, as well as their salts or esters, may be advantageously used in human and veterinary therapeutics for the treatment of affections requiring a high level of the heme oxygenase enzyme, in the prevention and the inhibition of the effects of stress on cells and tissues, and to prevent and treat tissue and organ transplant rejections. For example, their use at the indicated doses is particularly useful for the treatment of the affections listed here-after.
inflammations associated to type I and I diabetes, such as peripheral neuropathy and chronic skin ulcer,
macular degeneration
retinitis,
non-infectious intermediate or posterior uveitis
glaucoma
“connectivitis” (such as lupus erythematosus, scleroderma, sacoidosis)
erythema nodogum and adult atopic dermititis, in case of inefficacy of or intolerance or contra-indication to traditional treatments (phototherapy and/or photochemotherapy)
myasthenia and mucoviscidosis
chronic sinusitis, obstructive chronic bronchitis, bronchioectasy
tuberculosis (with its pulmonary, renal and bone localisations) and the prevention of the formation of granulomas (such as in parasitosis, leishmaniasis, pneumoconiosis)
chronic hepatitis (of viral or alcoholic origin), chronic pancreatitis
glomerulonephritis, the nephrotic syndrome and toxemia (<<uremia >>) of the patient under haemodialysis
opportunistic infections of immunosuppressed patients or patients burned to the 3rd degree, osteomyelitis
hemolytic anemia, sickle-cell anemia, hemarthrosis
atheromatous plaque development, hemorrhagic ictus, thrombophlebitis
acquired bone marrow failure (even if the patient had an allogenic bone marrow graft)
the prevention of tissue (cartilage, skin, bone marrow) or organ (liver, kidney, heart) graft rejection
the prevention of the graft rejection and of the rejection after the transplantation, including in the initial phase of the renal transplant, as well as the preventive or curative treatment of the graft-versus-host disease (GVHD)
the treatment of rejection, notably in patients who have initially been treated by immunosuppressive protocols.

Diacerein and rhein are both very slightly soluble in water and in alcohols, and are therefore preferably administered per oral route. The usual administration forms per oral route in the pharmaceutical area are appropriate, and for example, the drug can be administered under the form of tablets, capsules or soft gelatine capsules, or any other appropriate dosage form.

A particularly appropriate form of administration is the one described in patent EP 862423 describing the capsules or soft capsules in which diacerein is mixed with liquid oil and a non ionic surfactant, allowing of a good bioavailability to be obtained. Another form which can be used in the invention, is described in patent U.S. Pat. No. 6,124,358 and is prepared by comicronisation of rhein or diacerein with lauryl sulphate, for example sodium lauryl sulphate.

The posology is determined by the practitioner according to the state of health of the patient, but it is generally comprised between 25 mg and 500 mg per day, preferably between 50 mg and 100 mg per day. It is relatively independent from the weight of the patient, in adult subjects. The unitary doses, for oral administration, are generally comprised between 25 mg and 50 mg.

1. A method of treatment of an affection requiring an increase of the levels of the heme oxygenase enzyme in a human or animal subject comprising administering a therapeutically effective amount of rhein and rhein derivatives.

2. The method according to claim 1, wherein rhein and the rhein derivative are represented by the following general formula (I): 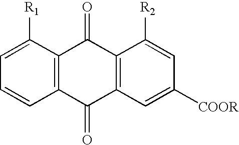
in which R represents a hydrogen atom, or an alkyl group, or an alkaline or hearth-alkaline metal atom, R₁ and R₂, identical or different, represent a hydroxyl group or an acyloxy group of formula R′ —COO— where R′ is an alkyl group of 1 to 4 carbon atoms.

3. The method according to claim 2, wherein the compound of formula I is rhein or diacerein.

4. The method according to claim 1, wherein the rhein or rhein derivative is administered at a dose of 25 to 500 mg per day.

5. The method according to claim 1, wherein the rhein or rhein derivative is administered per oral route.

6. A method of manufacturing a medicinal product for the treatment of an affection requiring an increase of the levels of the heme oxygenase enzyme comprising manufacturing rhein or rhein derivatives, wherein the medicinal product comprises rhein or rhein derivatives.

7. The method according to claim 6, wherein rhein and the rhein derivative are represented by the following general formula (I): 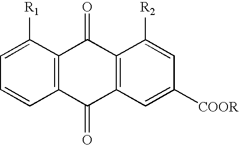
in which R represents a hydrogen atom, or an alkyl group, or an alkaline or hearth-alkaline metal atom, R₁ and R₂, identical or different, represent a hydroxyl group or an acyloxy group of formula R′′—COO— where R′ is an alkyl group of 1 to 4 carbon atoms.

8. The method according to claim 7, wherein the compound of formula I is rhein or diacerein.

9. The method according to claim 6, wherein the rhein or rhein derivative is administered at a dose of 25 to 500 mg per day.

10. The method according to claim 6, wherein the rhein or rhein derivative is administered per oral route.

11. The method according to claim 10, wherein the unitary dose is comprised between 25 mg and 50 mg.

12. The method according to claim 1, wherein the rhein or rhein derivative is associated with a non-steroidal anti-inflammatory drug (NSAID) or with a COX-2 inhibitor.

13. The method according to claim 1, wherein the affection requiring an increase of the levels of the heme oxygenase enzyme is transplant rejections.

14. The method according to claim 1, wherein the affection requiring an increase of the levels of the heme oxygenase enzyme is effects of stress on cells and tissues.
